(19) United States
(12) Patent Application Publication
(10) Pub. No.: US 2015/0265809 A1
(43) Pub. Date: Sep. 24, 2015

(54) GUIDE WIRE

(71) Applicant: ASAHI INTECC CO., LTD., Nagoya-shi (JP)

(72) Inventor: Kenichi MATSUO, Izumiotsu-shi (JP)

(21) Appl. No.: 14/630,732

(22) Filed: Feb. 25, 2015

(30) Foreign Application Priority Data
Mar. 20, 2014 (JP) 2014-57516

(51) Int. Cl. A61M 25/09

(52) U.S. Cl. CPC A61M 25/09; A61M 2025/09083

(57) ABSTRACT

A guide wire includes a coil body composed of a proximal-end coil and a distal-end coil connected to each other at a connecting portion in a manner that secures alignment of the two coils and sufficient penetration of a brazing metal into the connecting portion of the coils. The distal-end coil is formed of a first wire that is coiled, and the proximal-end coil is formed of a second wire that is coiled. The distal-end coil and the proximal-end coil are connected to each other with alternately disposed windings of the first wire and the second wire. Furthermore, the connecting portion of the coil body includes a gap. In this manner, the central axes of the two coils can be easily kept aligned with each other even before brazing, and a brazing metal or the like can easily penetrate into the connecting portion.

### CROSS REFERENCE TO RELATED APPLICATION

This application claims priority to Japanese Application No. 2014-57516 filed on Mar. 20, 2014, the contents of which are incorporated by reference herein in their entirety.

### BACKGROUND

The disclosed embodiments relate to a medical device. Specifically, the disclosed embodiments relate to a guide wire to be inserted into a lumen such as a blood vessel.

Guide wires used for insertion of catheters into blood vessels are known. In the insertion of a catheter, a guide wire is first inserted into a blood vessel, and then the catheter is moved along the guide wire. Thus, the guide wire acts as a guide for guiding the catheter to a lesion.

Guide wires typically include a core shaft and a coil body covering the core shaft. The coil body of the guide wire may include two kinds of connected coils. The two coils are connected by interposing the wires of one coil between the wires of the other coil, as in Japanese Unexamined Patent Application Publication No. S60-168466, Japanese Unexamined Patent Application Publication No. 2005-46603, and Japanese Unexamined Patent Application Publication No. 2006-297152. In this manner, a coil restoring force holds the wires of one of the coils between the wires of the other coil. Thus, even before a connecting portion of the two coils is bonded with a brazing metal, the central axes of the two coils are easily kept aligned with each other, advantageously facilitating assembly of the guide wire.

In the guide wire of Japanese Unexamined Patent Application Publication No. 2005-46603 and Japanese Unexamined Patent Application Publication No. 2006-297152, however, there are no gaps between wires in the connecting portion. This makes it difficult to apply a brazing metal into the connecting portion so as to obtain sufficient bonding strength. In the guide wire of Japanese Unexamined Patent Application Publication No. 560-168466, there is a large pitch between the two coils in the connecting portion so that gaps are formed between coil wires in the connecting portion. However, this makes it difficult to keep the central axes of the coils aligned before brazing, which in turn makes assembly of the guide wire more difficult.

### SUMMARY

As described above, in the related art, it is difficult to obtain both stable alignment of the two coils during assembly and sufficient penetration of a brazing metal into the connecting portion of the coils.

The disclosed embodiments have been devised to address the above problem of the related art. An object of the disclosed embodiments is to provide a guide wire having a coil body composed of two coils connected to each other in a manner that obtains both stable alignment of the two coils and sufficient penetration of a brazing metal into the connecting portion of the two coils.

In order to address the above problem, the guide wire of the disclosed embodiments includes a core shaft and a coil body that covers (i.e., is disposed around) the core shaft and includes a distal-end coil and a proximal-end coil. The distal-end coil and the proximal-end coil are connected to each other with alternately disposed windings of wires. Throughout this description, each winding of a wire may be called a “wire” for simplicity of reference. However, it should be understood that the distal-end coil and the proximal-end coil preferably are each formed by coiling a single wire. The proximal-end coil has a first wire (i.e., a first winding of the single wire forming the proximal-end coil) that is held by two of the wires of the distal-end coil (i.e., two adjacent windings of the single wire forming the distal-end coil) from both sides. The proximal-end coil also has a second wire (i.e., a second winding of the single wire forming the proximal-end coil) that is adjacent to the first wire and in contact with one of the wires of the distal-end coil from the proximal side of the second wire. Thus, even before a connecting portion between the two coils is bonded with a brazing metal or the like, the central axes of the two coils can be easily kept aligned with each other. This facilitates the connection of the coils during assembly of the guide wire,

The guide wire of the disclosed embodiments has a gap between the second wire of the proximal-end coil and the wire of the distal-end coil that is adjacent to a distal side of the second wire. This facilitates penetration of the brazing metal or the like into the connecting portion, sufficiently securing the bonding strength between the distal-end coil and the proximal-end coil, as well as the bonding strength between these coils and the core shaft.

As described above, the guide wire of the disclosed embodiments can obtain both stable alignment of the two coils during assembly and sufficient penetration of the brazing metal into the connecting portion of the coils.

In the guide wire of the disclosed embodiments, the wire that forms the proximal-end coil may be larger in diameter than the wire that forms the distal-end coil. This prevents the restoring force of the distal-end coil (the force that acts to restore the shape of the extended coil to an unextended state) from deforming the proximal-end coil. Thus, in the connecting portion of the two coils, gaps can be easily obtained between windings of the wire of the distal-end coil and windings of the wire of the proximal-end coil.

In the guide wire of the disclosed embodiments, the proximal-end coil may be made of a material having a larger modulus of elasticity than a material that forms the distal-end coil. This prevents the restoring force of the distal-end coil from deforming the proximal-end coil. Accordingly, even if the diameters of the wires forming the distal-end coil and the proximal-end coil are difficult to vary for design reasons (for example, in the design of a guide wire having an extremely small outside diameter), it is still possible to prevent the restoring force of the distal-end coil from deforming the proximal-end coil. Thus, in the connecting portion of the two coils, gaps between the wires of the distal-end coil and the proximal-end coil can be easily and reliably obtained.

### BRIEF DESCRIPTION OF THE DRAWINGS

FIG. 1 is an explanatory drawing showing the configuration of a guide wire according to the disclosed embodiments.

FIG. 2 is an enlarged view of a connecting portion of two coils in the guide wire of FIG. 1.

FIGS. 3A-3C are explanatory drawings illustrating the assembly of the guide wire of FIG. 1. FIG. 3A shows a step of brazing a distal-end coil to a distal end of a core shaft of the guide wire; FIG. 3B shows a step of connecting the distal-end coil and a proximal-end coil to each other by interposing wires thereof; and FIG. 3C shows a step of brazing a connecting portion between the distal-end coil and the proximal-end coil.

FIG. 4 is an enlarged view of a connecting portion between two coils in a guide wire where the wires forming the coils each have a different diameter.

FIG. 5 is an enlarged view showing a connecting portion of two coils in a guide wire where the wires forming the coils are each formed of a different material.

### DETAILED DESCRIPTION OF EMBODIMENTS

A guide wire according to the disclosed embodiments will be described below.

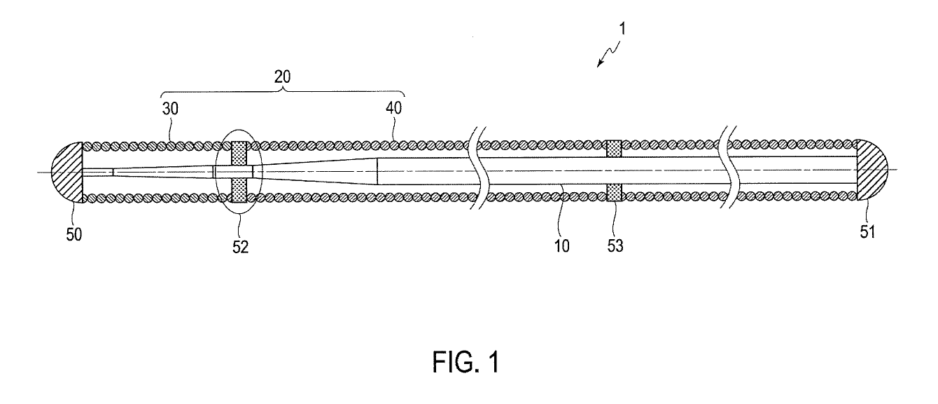

FIG. 1 is an explanatory drawing showing the configuration of a guide wire 1 according to the disclosed embodiments. As shown in FIG. 1, the guide wire 1 includes a core shaft 10 and a coil body 20 covering (disposed around) the core shaft 10.

The core shaft 10 is tapered to a decreasing size toward a distal end of the guide wire 1 in order to impart flexibility to the distal end of the guide wire 1. The coil body 20 includes two connected coils (a distal-end coil 30 and a proximal-end coil 40).

A distal end of the core shaft 10 and a distal end of the coil body 20 are joined to each other via a distal-end joint 50. A proximal end of the core shaft 10 and a proximal end of the coil body 20 are joined to each other via a proximal-end joint 51. Moreover, the core shaft 10 and the coil body 20 are joined to each other via a first intermediate joint 52 at a connecting point of the distal-end coil 30 and the proximal-end coil 40, and via a second intermediate joint 53 at an intermediate portion of the proximal-end coil 40.

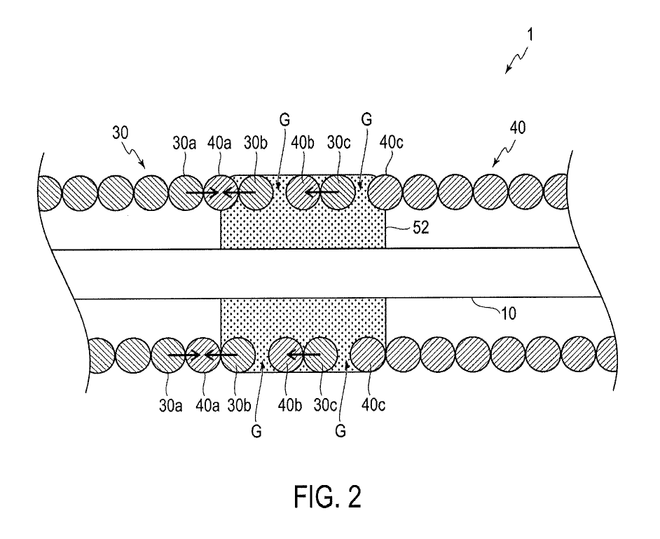

FIG. 2 is an enlarged view of the connecting portion of the two coils in the guide wire 1. As shown in FIG. 2, the two coils (the distal-end coil 30 and the proximal-end coil 40) are connected to each other by alternately disposing (interposing) the wires of the distal-end coil 30 and the wires of the proximal-end coil 40 (i.e., by interposing windings of the wire forming the distal-end coil and windings of the wire forming the proximal-end coil).

In the guide wire 1, a winding of the wire (first wire 40a) at the distal end of the proximal-end coil 40 is held between windings of the wire (wires 30a and 30b, positioned at both sides of the first wire 40a) of the distal-end coil 30. A second winding of the wire of the proximal-end coil (second wire 40b) adjacent to the proximal side of the first wire 40a is in contact with a winding of the wire (wire 30c) of the distal-end coil 30, the wire 30c being located on the proximal side of the first wire 40a.

Furthermore, in the connecting portion of the two coils, a gap G is provided between the second wire 40b of the proximal-end coil 40 and the wire 30b of the distal-end coil 30, the wire 30b being adjacent to the distal side of the second wire 40b. Another gap G may be provided between a third wire 40c of the proximal-end coil 40 and the wire 30c of the distal-end coil 30, the wire 30c being adjacent to the distal side of the third wire 40c.

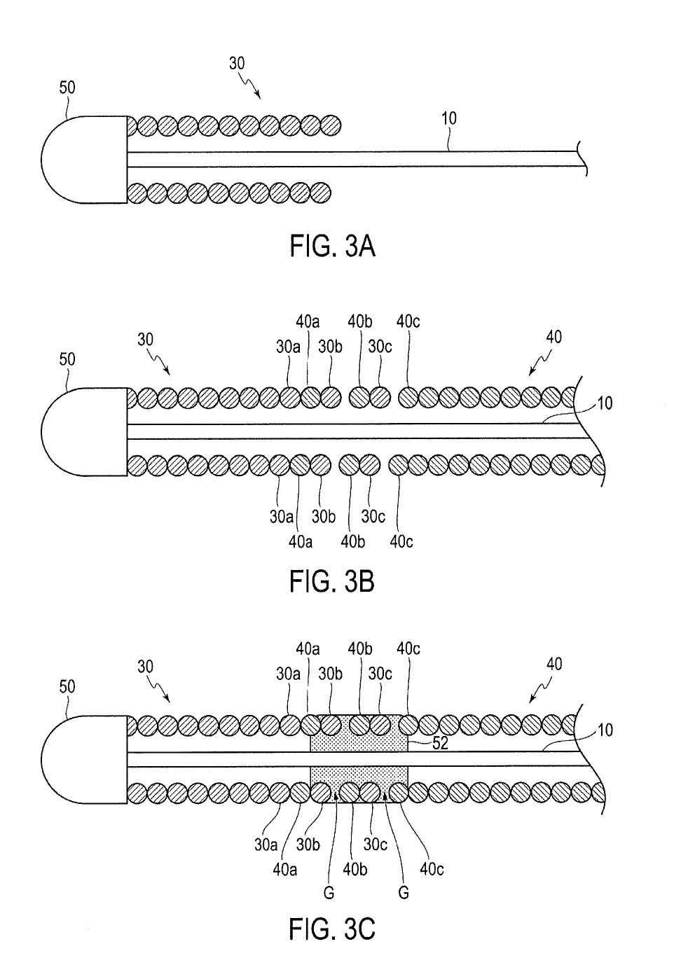

FIGS. 3A-3C are explanatory drawings illustrating assembly steps of the guide wire 1. In FIG. 3A, the distal-end coil 30 is brazed to the distal end of the core shaft 10 to form the distal-end joint 50. Subsequently, in FIG. 3B, the proximal-end coil 40 is inserted from the proximal end of the core shaft 10, and then the distal-end coil 30 and the proximal-end coil 40 are connected to each other by interposing windings of the wire forming the distal-end coil 30 and windings of the wire forming the proximal-end coil 40. In FIG. 3C, the connecting portion is brazed between the distal-end coil 30 and the proximal-end coil 40 at the first intermediate joint 52.

In the guide wire 1, the distal end of the proximal-end coil 40 has a relatively large pitch with respect to the proximal end of the distal-end coil 30 before the two coils are connected to each other. Thus, when the two coils are connected, the first wire 40a of the proximal-end coil 40 is held in place by wires (30a, 30b) of the distal-end coil 30 from both sides, and the second wire 40b adjacent to the proximal side of the first wire 40a is in contact with the wire 30c of the distal-end coil 30 from the proximal side of the second wire 40b (see FIG. 3B). Hence, even before brazing, the central axes of the two coils are easily kept aligned with each other. This eliminates the need for fastening techniques for temporarily fixing the central axes of the two coils, facilitating the connection of the coils and the overall assembly of the guide wire 1.

After the distal-end coil 30 and the proximal-end coil 40 are connected as shown in FIG. 3B, a brazing metal is applied to the connecting portion. As described above, the distal end of the proximal-end coil 40 has a relatively large pitch with respect to the proximal end of the distal-end coil 30 before connection. Thus, once the two coils are connected, the gap G is formed between the second wire 40b of the proximal-end coil 40 and the wire 30b of the distal-end coil 30, the wire 30b being adjacent to the distal side of the second wire 40b. Moreover, the gap G is formed between the third wire 40c of the proximal-end coil 40 and the wire 30c of the distal-end coil 30, the wire 30c being adjacent to the distal side of the third wire 40c. Thus, as shown in FIG. 3C, the brazing metal enters from the gaps G so as to form the first intermediate joint 52. Sufficient bonding strength is therefore obtained between the distal-end coil 30 and the proximal-end coil 40, as well as between these coils and the core shaft 10.

As described above, the guide wire 1 of the disclosed embodiments provides both stable alignment of the two coils (the distal-end coil 30 and the proximal-end coil 40) during assembly and sufficient penetration of the brazing metal into the connecting portion of the coils.

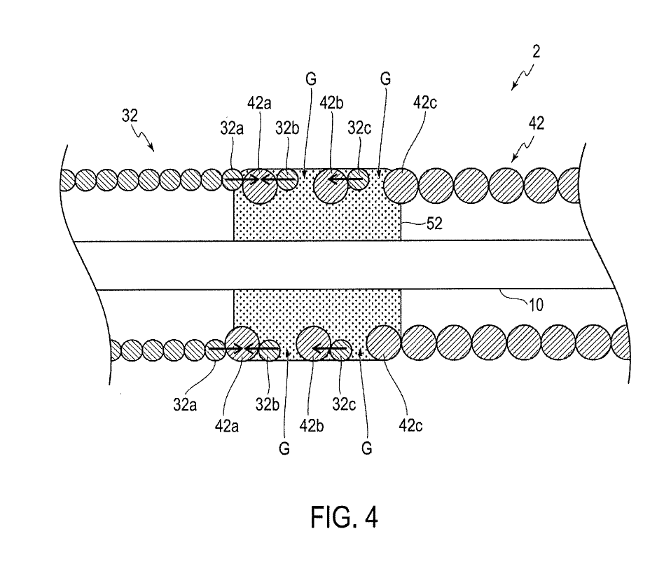

FIG. 4 is an enlarged view of a connecting portion between two coils in a guide wire 2 according to the disclosed embodiments. In the guide wire 2, a wire that forms a proximal-end coil 42 is larger in diameter than a wire that forms a distal-end coil 32.

Other features of the guide wire are identical to those of the guide wire 1. Specifically, a first wire 42a of the proximal-end coil 42 is held in place by wires (32a, 32b) of the distal-end coil 32 from both sides, and a second wire 42b adjacent to the proximal side of the first wire 42a is in contact with a wire 32c of the distal-end coil 32 from the proximal side of the second wire 42b. Moreover, in the connecting portion of the two coils, a gap G is provided between the second wire 42b of the proximal-end coil 42 and the wire 32b of the distal-end coil 32, the wire 32b being adjacent to the distal side of the second wire 42b. Another gap G may be provided between a third wire 42c of the proximal-end coil 42 and the wire 32c of the distal-end coil 32, the wire 32c being adjacent to the distal side of the third wire 42c.

The guide wire 2 provides both stable alignment of the two coils (the distal-end coil 32 and the proximal-end coil 42) and sufficient penetration of a brazing metal into the connecting portion of the coils.

The wire of the proximal-end coil 42 is larger in diameter than the wire of the distal-end coil 32, thereby preventing the restoring force of the distal-end coil 32 (the force that acts to restore the shape of the extended coil to an unextended state) from deforming the proximal-end coil 42. Thus, in the connecting portion of the two coils, the gaps G can be easily obtained.

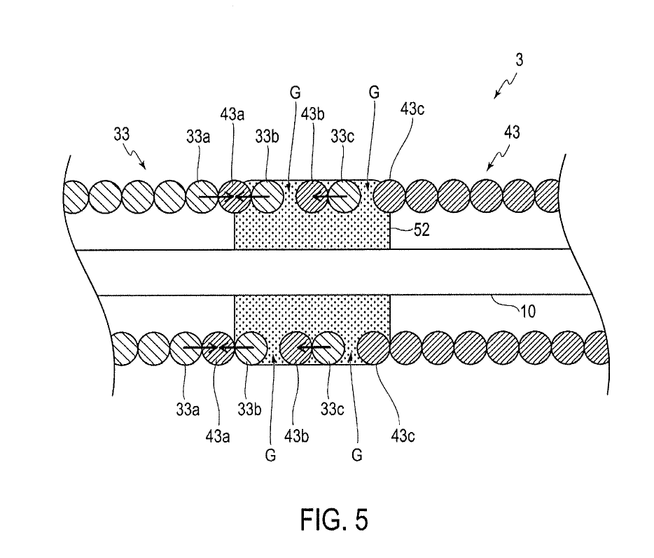

FIG. 5 is an enlarged view showing the connecting portion of two coils in a guide wire 3 according to the disclosed embodiments. In the guide wire 3, a proximal-end coil 43 is made of a material having a larger modulus of elasticity than a material of the distal-end coil 33. For example, the distal-end coil 33 may be made of platinum while the proximal-end coil 43 may be made of stainless steel having a larger modulus of elasticity than platinum.

Other features of the guide wire 3 are identical to those of the guide wire 1 (or the guide wire 2). Specifically, a first wire 43a of the proximal-end coil 43 is held in place by wires (33a and 33b) of the distal-end coil 33 from both sides, and a second wire 43b adjacent to the proximal side of the first wire 43a is in contact with a wire 33c of the distal-end coil 33 from the proximal side of the second wire 43b. Moreover, in the connecting portion of the two coils, a gap G is provided between the second wire 43b of the proximal-end coil 43 and the wire 33b of the distal-end coil 33 on the distal side of the second wire 43b. Another gap G may be provided between a third wire 43c of the proximal-end coil 43 and the wire 33c of the distal-end coil 33 on the distal side of the third wire 43c.

The guide wire 3 provides both stable alignment of the two coils (the distal-end coil 33 and the proximal-end coil 43) and sufficient penetration of a brazing metal into the connecting portion of the coils, like the guide wires of FIGS. 1 and 4.

The proximal-end coil 43 is made of a material having a larger modulus of elasticity than a material that forms the distal-end coil 33, thereby preventing the restoring force of the distal-end coil 33 from deforming the proximal-end coil 43. Accordingly, even if diameters of the wires forming the distal-end coil 33 and the proximal-end coil 43 are difficult to vary for design reasons (for example, in the design of a guide wire having an extremely small outside diameter), it is still possible to prevent the restoring force of the distal-end coil 33 from deforming the proximal-end coil 43. Thus, in the connecting portion of the two coils, the gaps G can be easily and reliably obtained.

Guide wires according to exemplary embodiments were described above. However, the present invention is not limited to these embodiments and can be implemented in various forms.

For example, in the guide wire 3, the proximal-end coil 43 is made of a material having a larger modulus of elasticity than the material of the distal-end coil 33 (see FIG. 5). In FIG. 5, the wire that forms the distal-end coil 33 and the wire that forms the proximal-end coil 43 are identical in diameter. However, the wire of the proximal-end coil may be larger in diameter than the wire of the distal-end coil (not shown).

In this configuration, the wire of the proximal-end coil is made of a material having a larger modulus of elasticity than the material of the wire forming the distal-end coil, and also has a larger diameter than the wire of the distal-end coil, thereby more reliably preventing deformation of the proximal-end coil. Hence, in the connecting portion of the two coils, the gaps between the wires of the distal-end coil and the proximal-end coil can be obtained with higher reliability, thereby securing sufficient bonding strength between the two coils (the distal-end coil and the proximal-end coil) and between these coils and the core shaft.

What is claimed is:

1. A guide wire comprising;
a core shaft; and
a coil body that covers the core shaft and includes a distal-end coil formed of a first wire that is coiled and a proximal-end coil formed of a second wire that is coiled, the distal-end coil and the proximal-end coil being connected to each other with alternately disposed windings of the first wire and the second wire,
wherein:
a first winding of the second wire at a distal end of the second wire is held in place between first and second windings of the first wire, and a second winding of the second wire located proximally of the first winding of the second wire is in contact with a third winding of the first wire from a proximal side of the second winding of the second wire, and
the coil body includes a first gap between the second winding of the second wire and the second winding of the first wire, the second winding of the first wire being adjacent to a distal side of the second winding of the second wire.

2. The guide wire according to claim 1, wherein the second wire that forms the proximal-end coil is larger in diameter than the first wire that forms the distal-end coil.

3. The guide wire according to claim 1, wherein the proximal-end coil is made of a material having a larger modulus of elasticity than a material that forms the distal-end coil.

4. The guide wire according to claim 2, wherein the proximal-end coil is made of a material having a larger modulus of elasticity than a material that forms the distal-end coil.

5. The guide wire according to claim 1, wherein the coil body further includes a second gap between a third winding of the second wire and the third winding of the first wire, the third winding of the second wire located proximally of the second winding of the second wire.

6. The guide wire according to claim 1, further comprising:
an intermediate joint formed of a brazed metal, wherein the brazed metal is disposed within the first gap in the coil body, the brazed metal contacting and interconnecting (i) the distal-end coil, (ii) the proximal-end coil, and (iii) the shaft.

7. A guide wire comprising:
a core shaft;
a coil body that is disposed around the core shaft and includes a distal-end coil and a proximal-end coil held together at a connecting portion by a restoring force of the distal-end coil; and
an intermediate joint formed of a brazed metal that connects the distal-end coil and the proximal-end coil to each other, wherein the brazed metal is disposed within a gap in the coil body at the connecting portion.

8. The guide wire according to claim 7, wherein:
the distal-end coil is formed of a first wire that is coiled and the proximal-end coil is formed of a second wire that is coiled, and
the distal-end coil and the proximal-end coil are connected to each other with alternately disposed windings of the first wire and the second wire.

9. The guide wire according to claim 7, wherein the intermediate joint further connects the coil body to the core shaft.

10. The guide wire according to claim 7, wherein the brazed metal is disposed within a plurality of gaps in the coil body at the connecting portion.

11. The guide wire according to claim 7, wherein a distal end of the proximal-end coil has a larger pitch than a proximal end of the distal-end coil when the distal-end coil is in an unextended state.
